(19) United States
(12) Patent Application Publication
(10) Pub. No.: US 2003/0186887 A1
(43) Pub. Date: Oct. 2, 2003

(54) Compounds for detection of infection by chlamydophila abortus

(76) Inventor: Garet Ewart Jones, Roslin (GB)

Correspondence Address: Lisa A Haile, Gray Cary Ware & Freidenrich, 4365 Executive Drive Suite 1100, San Diego, CA, 92121-2133, US

(21) Appl. No.: 10/182,763

(22) PCT Filed: Feb. 9, 2001
(86) PCT No.: PCT/GB01/00543

(30) Foreign Application Priority Data
Feb. 11, 2000 (GB) 0003217.7

(51) Int. Cl. A61K 38/10; C07K 7/08

(52) U.S. Cl. 514/13; 530/325; 530/326

(57) ABSTRACT

The present invention relates to compounds having binding affinity for specific sub-populations of antibodies to Chlamydophila abortus. More particularly, this invention relates to the use of compounds for the detection of Chlamydophila abortus infection. The proposed compound sequences provide unique reagents for the detection of sub-populations of antibodies to Chlamydophila abortus with supreme specificity with regard to possible cross-reactions with Chlamydophila pecorum and Chlamydophila psittaci in animals, such as sheep, goats and cattle, and Chlamydia trachomatis and Chlamydia pneumoniae in man.

The present invention relates to compounds having a binding affinity for one or more antibodies specific to Chlamydophila abortus and their use in the diagnosis of infection with Chlamydophila abortus. More particularly, this invention relates to the use of compounds for the detection of specific antibodies to C. abortus, which can be used diagnostically to identify C. abortus infections.

Members of the order Chlamydiales are obligate intracellular bacteria. This order has recently undergone a major expansion in its taxonomy (Everett et al., 1999), with current provision of four families. The largest family, the Chlamydiaceae, contains two genera, the Chiamydia and the Chlamydophila, and these currently comprise 3 and 5 species respectively. Infections with members of the family Chlamydiaceae in particular are widespread, both geographically and in terms of host range; they have been isolated from many species of host, including man, wild and domesticated birds, marsupials, ruminants, other mammals and invertebrates. Depending on the host and chlamydial species and subtype of species involved, infection with members of the Chlamydiaceae may result variously in pneumonia, enteritis, encephalitis, conjunctivitis, polyarthritis, abortions, genital disorders, generalised septicaemia and death; or be clinically inapparent.

The most important veterinary members of the genus Chlamydophila are Chlamydophila abortus, Chlamydophila psittaci and Chlamydophila pecorum. Chlamydophila abortus is the most common cause of abortion in sheep and goats in several countries including the United Kingdom, Greece and France. Cabortus has also been implicated as a cause of abortion, pneumonia and encephalitis in cattle (Holliman et al., 1994; Nabeya et al., 1991; Piercy et al., 1999) and, occasionally, pigs (Woollen et al., 1990) and humans (Buxton, 1986). Chlamydophila pecorum previously known as Chlamydia pecorum; Fukushi and Hirai, 1992) is even more widespread in ruminants than C. abortus and comprises several subtypes which differ in their pathogenicities, the diseases induced and the hosts infected. One subtype of C.pecorum is associated with an arthritis/conjunctivitis syndrome in sheep (Hopkins et al., 1973; Stephenson et al., 1973; Andrews et al., 1987): another with metritis in cattle (Wittenberg et al., 1993): another with encephalomyelitis in cattle (Harshfield, 1970): and another, ubiquitously distributed subtype, with an apparently symptomless, enteric infection of sheep (Clarkson and Philips, 1997).

Chlamydophila psittaci, which occurs primarily in birds, has also been reported to infect ruminants (Brown et al., 1988). Serological evidence presented later in this application indicates that such infection may be more common than is currently supposed.

The diagnosis of infection with chlamydiae is, by comparison with other bacterial infections or even some viruses, relatively difficult. In terms of detecting the agent, microscopic observation of the organism in infected tissues, excretions or faeces is used but is very insensitive. Culture requires the use of tissue culture cells or embryonated eggs, facilities not available to many laboratories and involving labour-intensive and time-consuming procedures. Several proprietary methods, such as the ‘Clearview’ (Unipath, Bedford, UK) and the ‘IDEIA’ (Dako Diagnostics, Ely, UK) are based on the use of antibodies (polyclonal and monoclonal) to detect specific antigen(s). These are usually effective, though may suffer from a relatively high false positive detection rate. More importantly, they require the collection of good quality samples from the field, which is often not possible. Finally, DNA detection methods (polymerase and ligase chain reactions), though extremely sensitive, are also expensive and therefore rarely applied in the veterinary field. However, perhaps the greatest drawback of antigen detection methods in veterinary diagnostics is that they cannot be applied to large numbers of samples in a rapid or automated system.

In contrast, tests with a veterinary application that detect specific antibodies have several advantages over tests that detect the agent. One is that the sample used, blood (or its derivatives, serum or plasma) is readily available and collectable throughout the year. Another is that large numbers of animals can be screened at relatively modest cost. Finally, serological tests enable the detection of carrier animals, which remain infected and infective after suffering disease, but which are difficult to detect using antigen detection techniques since they generally excrete the organism in question only under certain physiological conditions and in small numbers. Accordingly, antibody detection tests are the most favoured form of identifying individual animals or flocks infected with any given micro-organism. Much attention has been given to the development of serological tests for the diagnosis of chlamydial infections, particularly in sheep and goats, because of the difficulties associated with achieving a diagnosis through detection of the agent (Sting and Hafez, 1992; Markey et al., 1993; Sanderson et al., 1994; Martin, 1995; Anderson et al., 1995; Griffiths et al., 1996; Jones et al., 1997). The most commonly used serological test has been the Complement Fixation Test (CFT; Stamp et al., 1952), but many others have been developed, including Enzyme-Linked ImmunoSorbent Assays (ELISA) of both indirect and competitive type, Immuno-Fluorescent Assays (IFA) of both direct and indirect type, and Western blotting. Most tests hitherto described for chlamydial antibodies have used as antigen some form of the chlamydial organism, either as a complete, relatively untreated entity (IFAs), boiled (CFT), or as extracts in which some components of the whole organism have been eliminated or greatly reduced by chemical treatment (some ELISAs). One novel test has used a recombinant form of a major antigen of the Chlamydiaceae family, namely the lipopolysaccharide (Brade et al., 1994; Griffiths et al., 1996).

None of the tests described hitherto has achieved the specificity necessary in veterinary applications to distinguish between infections with C.abortus, C.pecorum and C. psittaci. This is because C. pecorum and C. psittaci share several antigenic determinants in common with C. abortus.

The term “antigen” refers to an entire molecule, part(s) of which are capable of inducing the formation of antibodies when infecting, or injected into, an animal or man. The term “epitope” refers to a precise part of an antigen which induces antibody formation. An epitope generally comprises 3 to 10 amino acids, either in straight line (“linear” “sequential” or “continuous”) formation or clustered together through the folding of the complete protein molecule into “conformational” (or “assembled” or “discontinuous”) epitopes. Defining the three-dimensional structure and topographical relationships of amino acids in a conformational epitope is usually not possible at present. An antigen usually possesses several epitopes, each of which induces the formation of a different sub-population of antibodies in the infected or recipient host.

The epitopes shared by C. abortus, C. pecorum and C. psittaci are found on several important antigens, in particular lipopolysaccharide, the Major Outer Membrane Protein (MOMP) and a heat-shock protein of approximately 60 kDa in size (“60hsp” or “hsp60”). Thus, conventional serological tests that use preparations of “native” material (i.e. produced by the micro-organism itself) containing MOMP, lipopolysaccharide and/or the 60 hsp protein of C. abortus as antigen(s) will lack specificity between C.abortus, C. pecorum and C.psittaci. One solution to this is to use a competitive format in which monoclonal antibodies are used to target specific epitopes. However, competitive serological tests are often cumbersome and difficult to standardise, require high quality monoclonal antibodies and target only one epitope unless several monoclonal antibodies are available to detect different, specific epitopes.

Another solution to the problem of serological test specificity is to use synthetic peptides as antigens. For example, peptide-based tests are now commercially available for the diagnosis of human infections involving C. trachomatis (Narvanen et al., 1996; Paukku et al., 1996; WO 99/00414; U.S. Pat. No. 5,516,638; U.S. Pat. No. 5,629,167). However, one of the drawbacks in the use of single epitope assays is that specificity is often obtained at the expense of sensitivity since, in a given population, only a proportion of animals will be capable of recognising a single epitope as foreign and therefore produce antibodies against it. Thus their use will also often result in a marked and unacceptable loss of sensitivity, since each peptide detects only one sub-population of antibodies out of the tens, hundreds or thousands induced by infective organisms. Furthermore, the very small size of peptides (a 20-mer is typically of approximately 2400 Mol. Wt.) means that they are less capable of retaining integrity of shape compared with the full antigenic molecule.

Thus, a method of detecting of C. abortus infection with high specificity and sensitivity, using a procedure which is simple and reliable, is still lacking in the art.

According to a first aspect of the present invention, there is provided a compound having a binding affinity for one or more antibodies specific to Chlamydophila abortus, having the following general formula:

(X)ₘ-[A]-(X)ₙ-[A]ᵥ-(X)ₚ-[A]_{w}-(X)_{q}-[A]_{y}-(X)ᵣ-[A]_{z}-(X)ₛ

wherein

X is a linker molecule;

m, n, p, q, r and s represent the number of linker molecules and are independently any integer between 0 to 15;

v, w, y and z are independently 0 or 1; and

A is independently selected from the following sequences:

i. TAAANYK

ii. GTAAANYK

iii. TAAANYKT

v. GTAAANYKT

v. KGSSIAAD

vi. KGSSIAADQ

wherein the compound does not comprise:

PTGTAAANYK, VKGSSLKADQ, LVGLIG, AFN, LP, TPT, TG, VK;

and the compound is not GSSIAADQ.

The present invention provides compounds for the detection of specific sub-populations of antibodies to C. abortus which provide not only specificity, particularly with regard to distinguishing antibodies against C. abortus from those against C. pecorum and C. psittaci, but also an acceptable level of sensitivity. The compounds are preferably highly specific to antibodies to C. abortus.

The above-defined compounds comprise epitopic sequences, which induce the formation of antibodies to C. abortus and to which the said antibodies bind as part of the natural antigen, ie MOMP. The sequences are from the four variable regions which are found in the MOMPs of chlamydiae. The term “peptide” is used herein in a broad sense to indicate that a particular molecule comprises a plurality of amino acids joined together by peptide bonds. It therefore includes within its scope substances, which may sometimes be referred to in the literature as peptides, polypeptides or proteins (whether or not they are covalently bound to other moieties—e.g. to form fusion proteins).

Preferably the compound of the present invention consists of 18 to 24 amino acids. An enhanced degree of reactivity is usually obtained with this length compared with shorter constructs. More preferably, the compound consists of 20 to 23 amino acids.

The length of the compounds of the present invention may be altered using one or more linker molecules. The linker molecule may comprise amino acid residues which are considered not to contribute directly to the epitope reactivity. For example, a single amino acid may be used, such as serine, lysine, glycine, asparagine, tyrosine or arginine. Glycine, asparagine, serine and lysine are preferred. A linker comprising more than one amino acid, such as a serine-glycine motif, may also be used. An example of a compound comprising linker molecules is as follows:

KKKKKKKKKKKGSSIAAD

(For this compound, X in the above general formula is Lysine (K); m is 10; n, p, q, r and s are zero; A is “KGSSIAAD”; and v, w, y and z are zero)

The linker molecule may also be, for example, biotin, which may act as both a spacer and as an attachment link to, for example, streptavidin.

The compounds of the present invention may also involve repetitions of a single peptide epitope or chimeras of peptide epitopes. Such compounds may have uninterrupted sequences of the above-defined peptides. Thus, in the above general formula, n, m, p, q, r and s may all be zero. Examples of such compounds are;

1) GTAAANYKTGTAAANYKT

2) KGSSIAADQKGSSIAADQ

Separation of epitopes within a compound with a linker has the advantage of ensuring better epitope definition. The linker molecule may also be the amino acids found in the natural sequence of the represented Variable Segment. Three or more of such amino acids may represent ancillary epitopes.

Preferably the compounds of the present invention are dimers; ie v is 1 and w, y and z are all 0.

Preferably A is peptide (v) above (GTAAANYKT) or peptide (vi) above (KGSSIAADQ).

Preferably the compound has the formulae:

[X]ₙ-GTAAANYKT-[X]ₙ-GTAAANYKT;

wherein X is S (serine) or K (lysine); or

[X]ₙ-KGSSIAADQ-p[X]ₙ-KGSSIAADQ;

wherein X is G (glycine) or N (asparagine).

Specific examples of compounds of the present invention are:

Compound #1: SGTAAANYKTKGTAAANYKT

Compound #2: GKGSSIAADQNKGSSIAADQ

It will be recognised by the skilled person that, within the peptides defined above, substitutions and/or deletion of one or possibly more of the amino acids constituting the compound can be made without excessive decrease in the reactivity and/or specificity of the epitope. Such variants of the peptides described above, which are functionally equivalent to the present compounds but contain certain amino acid residues which may be non-naturally occurring, modified and/or synthetic, are within the scope of the present invention, if they are recognised by antibodies specific to C. abortus. Such derivatives/homologues form another aspect of the invention. The skilled person would be aware that the antibody binding ability of epitope analogues containing, for example, single amino acid substitutions, may be determined using a suitable scanning technique.

The skilled person is aware that various amino acids have similar properties. One or more such amino acids of a peptide can often be substituted by one or more other such amino acids without eliminating a desired activity of that peptide. For example, the amino acids glycine, alanine, valine, leucine and isoleucine can often be substituted for one another (amino acids having aliphatic side chains).

Other amino acids that can often be substituted for one another include:

phenylalanine, tyrosine and tryptophan (amino acids having aromatic side chains); lysine, arginine and histidine (amino acids having basic side chains); aspartate and glutamate (amino acids having acidic side chains); asparagine and glutamne (amino acids having amide side chains); and cysteine and methionine (amino acids having sulphur containing side chains).

Substitutions of this nature are often referred to as “conservative” or “semi-conservative” amino acid substitutions. Amino acid deletions and insertions relative to a peptide as defined above can also be made.

Peptides incorporating amino acid changes (whether substitutions, deletions or insertions) relative to the sequence of an epitope as defined above can be provided using any suitable techniques. The compounds of the present invention can be prepared by conventional processes for synthesising peptides (Shroder and Lubke, 1966), by solid phase peptides synthesis (Merrifield and Barany, 1980), or any other suitable technique for peptide synthesis.

Such techniques generally utilise solid phase synthesis. Chemical synthesis techniques that allow peptides having particular sequences to be produced have now been automated. Apparatus capable of chemically synthesising polypeptides is available, for example, from Applied Biosystems. If necessary, short peptides can be synthesised initially and can then be ligated to produce longer peptides. The compounds may also be produced as larger fusion proteins, comprising more than one of the above-defined peptides, of the same type or different type, which are then split up by appropriate techniques.

Additional amino acids or other moieties may be attached to either ends of the compound. For example, the compounds may be conjugated at the amine terminus with biotin. As described below, the strong affinity of biotin for avidin and streptavidin enables the attachment of the compounds to solid phase supports such as microtitration plates or latex particles. This may be useful in certain embodiments of the present invention described more fully below. Other possible modifications to the compounds include NH₂-acetylation and COOH-terminal amidation.

A compound of the present invention may be provided in substantially pure form by using the aforesaid techniques. Thus the present invention also provides a composition comprising one or more compounds as set out in the first aspect. The compounds in the composition may be the predominant component present (i.e. where it is present at a level, when deterimined on a weight/weight basis, of more than 50% of the total composition; preferably at a level of more than 75%, of more than 90%, or of more than 95% of the total composition).

The compounds of the present invention may be specifically immuno-reactive with antibodies to C. abortus, in the presence of antibodies against other Chlamydophila species e.g. Chlamydophila pecorum, Chlamydophila psittaci, Chlamydia trachomatis and Chlamydophila pneumoniae. The skilled person is able to determine whether or not a particular compound has a binding affinity for one or more antibodies to C. abortus, by using techniques described herein and which are known in the art.

The use of peptides in ELISAs for C. abortus antibodies has been described in the scientific literature, although commercial kits using these peptides have not been produced (Kaltenboeck et al., 1997; Salti-Montesanto et al., 1997). Neither of these however indicated the specificity or sensitivity of the peptides described. For example, neither group of workers determined the specificities of the epitopes described herein, using antisera against other species of Chlamydophila.

Thus, a feature of this invention is the recognition that the epitopes used to diagnose C. abortus infection should be defined to exclude epitopes which are cross-reactive with antibodies induced by other species of Chlamydophila. Surprisingly, it has been found that cross-reactivity with other species of Chlamydophila may be caused by the presence of as little as one amino acid on the N- or C-terminal of the epitopic sequences defined herein.

In contrast to previous attempts to detect antibodies specific to C. abortus, the present invention provides specificity as well as sensitivity.

In the second aspect, the present invention provides the use of a compound as set out in the first aspect of the invention in medicine. The term “medicine” used herein includes the field of veterinary medicine. The invention finds particular use in the diagnosis of infection of a subject by C. abortus, more specifically the detection of an antibody against C. abortus in a biological sample derived from the subject.

Preferably the subject is a mammal, for example, a human, sheep, cattle, goat, pig or horse. Domestic pets are also within the scope of the invention.

The biological sample may be blood, saliva, mucus or other body fluid, tissue or any solubilisable sample which might contain antibodies to C. abortus.

In the third aspect, the present invention provides a method of detecting an antibody to C. abortus in a biological sample, comprising the steps of:

contacting the sample with one or more compounds as defined in the first or second aspect of the invention; and

detecting, if any, the binding of said antibody to said compound.

The sensitivity of a serological test is often sacrificed when a high degree of specificity is required, since specificity implies a selection and hence a corresponding reduction in the number of epitopes used, and therefore the number of subpopulations of antibodies for which examination is made. To provide a greater number of reactive sites and, thus, to improve sensitivity while maintaining specificity, the compounds are preferably used together.

Detection and measurement of the number of antibodies remaining adherent to their specific antigen (i.e. synthesised compound containing at least one epitope), if any, provides a means by which exposure to infection with C. abortus may be diagnosed. The compounds of the present invention can be used either on their own or in combination. It is preferred that more than one type of compound, preferably two, is used. The sensitivity of the test, as defined by the proportion of positive results by test in respect to a true number of positives, depends greatly on the composition and number of compounds used and the method by which they are presented to the sample immunoglobulins. The compounds used herein are preferably highly specific in respect to antibodies to C. abortus.

In a preferred embodiment, the method comprises contacting the sample with a combination of Compound #1 and Compound #2:

Compound #1: SGTAAANYKTKGTAAANYKT

Compound #2: GKGSSIAADQNKGSSIAADQ

The compounds of the present invention may be used to detect antibodies to C. abortus in various test systems, including homogeneous and heterogeneous binding immunoassays. These include the enzyme-linked immunosorbent assay (ELISA) (indirect and competitive forms), haemagglutination assay, radioimmunoassay, latex agglutination assay, fluorescent polarising assay, biosensors of various types, including amperometric, electrochemical and other means for detecting antibodies or complexes thereof, including immunological complexes, and others.

The compounds can also be coupled to other compound(s), to a large carrier protein, to solid support, latex particles or bound in another way. The compounds may be bound in such a way as to keep their antigenic integrity. As described above, to couple the compounds to a solid support or another molecule, additional amino acids or other moieties may be attached to the amine end of the compound. In particular, it is preferred that the compounds are conjugated at the amine terminus with biotin. The strong affinity of biotin for avidin and streptavidin enables the attachment of the compounds to solid phase supports such as microtitration plates or latex particles by previous coating of such supports with avidin or streptavidin. The compounds may also be modified by NH₂-acetylation and/or COOH-terminal amidation.

The compounds may be attached to a solid support. Thus, in an ELISA system the compounds are attached to the solid phase, the ELISA plates being modified for attachment of compounds so as to maintain the antigenic integrity of the compounds. The attachment system to solid supports preferably uses the strong bonding between streptavidin and biotin. Streptavidin is coated onto the solid support e.g. ELISA plate, and the compounds are biotinylated at the amine end, in accordance with standard laboratory procedures. One alternative to using streptavidin-biotin as the means of attaching compounds to the solid phase is to use a proprietary system which employs activated dextran or similar polymer; such a system is represented by Aquabind™ plates [M&E, Denmark]. In this case, preferably the first compound residue [at the amine end] which is attached to the solid phase is lysine.

Not all methodologies described in the literature are effective in attaching the compounds described in this application to the solid phase, specifically ELISA plates.

For example, the systems described by Niveleau et al. (1995) and by Ball et al. (1994) were found to be ineffective in binding Epitope 2 when presented as an unmodified 8-mer.

Solid supports include, but are not limited to, polystyrene-or polyvinyl microtiter plates, glass tubes or glass beads, chromatographic supports such as paper, cellulose, cellulose derivatives and silica. Carrier proteins include bovine serum albumin and enzymes such as horseradish peroxidase.

Compounds may be attached indirectly to a solid phase to retain their antigenicity. Thus, the attachment may be performed, for example, via an intermediate molecule, or any other spacer. Different techniques of attachment of compounds to a solid surface have been described (Cass and Ligler, 1998).

In another aspect of the invention, a diagnostic kit for infection by C. abortus is provided, comprising:

one or more compounds as defined in the first or second aspect of the invention; and

at least one reagent for detecting, if any, the binding of said antibody to said compound.

The reagent for detecting the formation of the immunological complex may comprise one or more of the following:

1. Protein G and/or Protein A

2. Monoclonal antibody or part thereof (F_{c} or F_{ab} region), and/or polyclonal antibody

The polyclonal and monoclonal antibodies may be to sheep or goat immunoglobulin. The polyclonal antibody may be raised in, for example, rabbits, pigs or horses.

The reagents may be conjugated to a suitable enzyme, such as horseradish peroxidase or alkaline phosphatase, in accordance with standard laboratory procedures.

Preferred features of each of the aspects apply to all other aspects of the invention.

The invention will now be illustrated using examples, which are, however, not limiting.

The following methods and materials were used in the experiments.

1. Peptide Synthesis

Synthetic peptides manufactured according to accepted practice (e.g. User's Manual for Peptide Synthesiser Model 430A, Applied Biosystems, 1986) were obtained from companies offering this service (e.g. Sigma Genosys Biotechnologies (Europe) Ltd., London Road, Pampisford, Cambs, UK: Cambridge Research Biochemicals, Gadbrook Park, Northwich, Cheshire, CW9 7RA). The peptides were obtained in lyophilised form as greater than 75% pure (confirmed by mass spectroscopy and HPLC) and biotinylated at the amine terminus.

2. Stock Peptide Solution Preparation

Stock solutions are prepared from the lyophilised peptides by re-suspending them to a concentration of 1 mg/ml. Depending on the peptide, resuspension requires either deionised, distilled water (DDW), 0.1% trifluoroacetic acid in DDW, acetic acid (various concentrations), acetonitrile, dimethylformamide or dimethyl sulphoxide, or combinations of these. The preferred solvent for Compounds #1 and #2 is water. Stock solutions are aliquoted in 0.5 ml volumes and maintained at a temperature below −15° C. A mixture of Compounds #1 and #2 is prepared by dissolving the peptides simultaneously in a suitable buffer, preferably carbonate/bicarbonate buffer at pH 9.6 (Appendix). The optimal concentration of Compound #1 in this mixture was determined to be 80 ng per ml and of Compound #2 was 140 ng per ml.

3. Preparation of Microtitration Plates for ELISA Assay

In the Applicant's experience at least two different forms of microtitration plates may be used. One form uses tresyl-activated dextran, which is applied to the polystyrene plate and forms a layer between the plate and the peptide (Gregorius et al., 1995). The commercially available plates (‘Aquabind™’) are produced by M&E, Denmark and marketed in the UK through Labsystems by Life Sciences. The peptides used for this form of plate are not biotinylated or modified in any form, but in the Applicant's experience, attachment of the peptide to the plate is enhanced by provision of a lysine residue at the amine terminus. The second form of plate is one modified by coating with streptavidin. Such plates are commercially available e.g. Labsystems, Pierce and Warriner. Alternatively, uncoated microtitration plates may be treated with streptavidin, preferably at a concentration of 10 μg per ml in carbonate-bicarbonate buffer (Appendix). The preferred microtitration plate types used to bind streptavidin are those modified to provide high protein binding e.g. Dynex Immulon 2, Greiner High Bind. To each well is added streptavidin in 150 μl volumes. The plates are incubated, preferably for 2 hours at 35-37° C., then the contents ejected and the plates washed 3 times with PBS buffer, pH 7.4 (Appendix). The plates are thoroughly dried and stored at 4° C. until required. Application of the peptide mixture is performed as follows:

The stock solutions (1 mg per ml) of the selected peptides are diluted in the same volume of carbonate-bicarbonate buffer, pH 9.6, to their optimum concentration. Preferably, Compounds #1 and #2 are used at 80 μg per ml and 140 μg per ml respectively. The peptide mixture solution is added in volumes of 150 μl per well. In a ‘controlled’ test, the peptide mixture is added to all wells in even numbered columns, while carbonate-bicarbonate buffer is added to all wells in odd-numbered columns in the same volume per well. In ‘screen’ tests, where control wells are not used for each sample, the peptide mixture is added to all 96 wells on the plate. The plates are incubated, preferably at 4° C. for 4 to 12 hours, then washed 4 times with PBS/Tween buffer (Appendix) and thoroughly dried, preferably for 4 hours in a fan-driven cabinet. The coated plates are placed in watertight bags with a desiccant, and the bags sealed and stored at 4° C. until required.

4. ELISA Methods

Plates coated as in Example 3 with Compounds #1 and #2, used as a mixture at 80 ng/ml and 140 ng/ml respectively, were used to test serum samples obtained from sheep which had been experimentally infected with C. abortus, sheep known never to have experienced infection with C. abortus and sheep which had been vaccinated with different strains of C. abortus or various subtypes of C. pecorum. Routinely, it is recommended that where there are large numbers of sera to be tested, an initial assay be performed in a ‘screen’ plate i.e. one in which all wells have been coated with peptide. Included on each test plate are standard serum samples obtained from sheep which had been challenged with C. abortus and which subsequently exhibited clinical signs of disease, accompanied by the development of high antibody titres against C. abortus. Two dilutions of standard serum are used. One, employed at a dilution of 1 in 1000, is used to indicate successful performance of the test (‘standard positive’). The other, employed at a dilution of 1 in 7000, is used to indicate a positive reaction (‘cut-off’ standard): all animals yielding a colour development (optical density or OD) which is higher than the OD shown by the cut-off standard are deemed to be positive. In the kits, the positive and cut-off standards are provided pre-diluted to working strength in a proprietary stabilisation buffer (Dako) which contains a preservative (ProClin 300; Supelco; used at 1 in 2000), and are not diluted further before addition to the test plate.

The test serum samples are diluted 1:100 in Serum Conjugate Diluent (Appendix). The test kits provide a Diluter plate, which has very low protein binding properties. By adding 15 μl of serum to 135 μl of Serum Conjugate Diluent on the Diluter plate, an initial 1 in 10 dilution is achieved. Transfer of 15 μl from the Diluter plate to 135 μl of Serum Conjugate Diluent dispensed into each well in the test plate enables a further dilution to 1 in 100 (from the original sample) on the test plate. This method has been shown to provide accurate and reproducible dilutions. Dilution of sera can also be performed, if preferred, in bijoux or tubes before their addition in 150 μl volumes to the Test plate.

Test and standard sera are incubated on the Test plate for 15 min at room temperature (˜20° C.), then 30 min at 37° C. Plate contents are then ejected and the plate washed 4 times with PBS/Tween (Appendix) and dried.

A probe to detect the presence of immunoglobulin, to which is conjugated an enzyme (the ‘conjugate’), is then added. Preferably, the probe constitutes Protein G (recombinant form, in which the moiety that binds to albumin has been eliminated; available from Sigma and Calbiochem) conjugated with horse radish peroxidase, but many forms of conjugate are available. These include, as probes, Protein A; and monoclonal or polyclonal antibodies against the immunoglobulins of the species providing the test sample. A commonly used alternative enzyme for conjugation to the probe is alkaline phosphatase. However, recombinant Protein G conjugated with horse radish peroxidase has been found to be particularly effective, in terms both of sensitivity and of providing a low background colour development i.e. the positive signal to ‘noise’ ratio is very high. The conjugate is diluted to appropriate concentration (generally 1 in 4000 to 1 in 5000) in Serum Conjugate Diluent (Appendix) and added to all wells in 150 μl volumes. The plate is incubated at 37° C. for 30 min, then the contents ejected, the plate washed 4 times with PBS/Tween (Appendix) and dried. Substrate, pre-warmed to room temperature for 30 min, is then added in 150 μl volumes to all wells. Preferably, the substrate comprises ABTS (2,2′-azino-di[3-ethyl-benzthiazoline sulfonate(6)]), although TMB (3,3′, 5,5′-tetramethylbenzidine) or OPD may also be used. Both substrates are available from several sources including Kirkegaard and Perry Laboratories, Neogen Corporation and Pierce and Warriner. The plate is incubated at room temperature for 30 min, then stop solution (sodium lauryl sulphate, 1.5% for ABTS; 0.2M H₂SO₄ for TMB) is added in 50 μl volumes to all wells. The plates are read (using a 405 nm or 450 nm filter respectively for ABTS and TMB) in a spectrophotometer specifically designed for the purpose i.e. an ELISA reader such as the Dynex MRX.

Following the screen test, it is recommended that all animals which yield an OD higher than the cut-off standard be re-tested on a control plate i.e. one in which each serum sample is added to two wells, one coated with peptide and one treated with carbonate-bicarbonate buffer only. This controls for a small proportion of animals which produce sera that stick non-specifically to polystyrene plates, thus causing false positive reactions. In controlled plates, the control well OD value is subtracted from the peptide-coated well OD value.

5. Preparation of 20×PBS

Reagents

To prepare 5L;

| | |
|---|---|
| Reagent | Quantity |

| | | |
|---|---|---|
| Sodium chloride (NaCl) | 800 | g |
| Potassium chloride (KCl) | 20 | g |
| Di-sodium hydrogen orthophosphate (Na₂HPO₄) | 11.5 | g |
| Potassium di-hydrogen orthophosphate (KH₂PO₄) | 20 | g |
| Reagent grade water | 5 | L |

Procedure

1. Measure water into flask using measuring cylinder

2. Add magnetic stirrer and place flask on heated stirrer set to 40° C.

3. Add reagents in order

4. Place foil cap over flask neck or lid onto bottle

5. Label with date, batch number and expiry date

6. Store room temperature

6. Preparation of 1×PBS/Tween

Reagents

To prepare 10L;

| | |
|---|---|
| Reagent | Quantity |

| | | |
|---|---|---|
| 20 × PBS | 500 | ml |
| Tween 20 (0.05% final concentration) | 5 | ml |
| Reagent grade water | 9.5 | L |

Procedure

1. Measure 20×PBS into container

2. Add 9.5L water

3. Add Tween using a 5 ml pipette. Flush through pipette with the PBS to remove all the Tween 20

4. Shake container well

5. Label with date, batch number and expiry date (1 year from production)

6. Store at room temperature

7. Preparation of Solution A (0.2M)

Reagents

To prepare 100 ml;

| | |
|---|---|
| Reagent | Quantity |

| | | |
|---|---|---|
| Anhydrous sodium carbonate (Na₂CO₃) | 2.12 | g |
| Reagent grade water | 100 | ml |

Procedure

1. Weigh out required amount

2. Measure water into flask using measuring cylinder

3. Add magnetic stirrer and dissolve

4. Label with date, batch number and expiry date (one month from production)

5. Store at room temperature

8. Preparation of Solution B (0.2M)

Reagents

To make 200 ml;

| | |
|---|---|
| Reagent | Quantity |

| | | |
|---|---|---|
| Sodium hydrogen carbonate (NaHCO₂) | 3.36 | g |
| Reagent grade water | 200 | ml |

Procedure

1. Weigh out NaHCO₂ and place in beaker

2. Add water to beaker

3. Add magnetic stirrer and dissolve

4. Label with date, batch number and expiry date (one month from production)

5. Store at room temperature

9. Production of 1×Carbonate/Bicarbonate (CO₃/HCO₂) Buffer

Reagents

To produce 100 μm;

| | |
|---|---|
| Reagent | Quantity |

| | | |
|---|---|---|
| Solution A | 5.25 | ml |
| Solution B | 22.25 | ml |
| Reagent Grade water | 72.5 | ml |

Procedure

1. Measure water using measuring cylinder and add to beaker

2. Measure out required amounts of solutions A and B into flask using pipettes

3. Mix well

4. Check pH; reject if not within range pH 9.3-9.9

5. Label with date, batch number and expiry date (1 week from production)

6. Store at 4° C.

10. Production of Serum and Conjugate Diluent

Reagents

To prepare 100 ml;

| | |
|---|---|
| Reagent | Quantity |

| | | |
|---|---|---|
| 1 × PBS/Tween | 100 | ml |
| Dried Milk (Cadburys Marvel ™) | 2.5 | g |

Procedure

1. Measure PBS/Tween into a bottle using a measuring cylinder

2. Add dried milk and mix well

3. Label with date, batch number and expiry date (one week from production)

4. Store at 4° C.

11. Protocol and Record for the Preparation 20× Stop Solution (30% Sodium Lauryl Sulfate)

Reagents

To prepare 200 ml;

| | |
|---|---|
| Reagent | Quantity |

| | | |
|---|---|---|
| Sodium lauryl sulfate (SLS) | 60 | g |
| Reagent grade water | 140 | ml |

Procedure

1. Measure water into beaker using measuring cylinder

2. Add SLS to water, place in 56° C. water bath and stir until fully dissolved

3. Pour into a 250 ml measuring cylinder and make up volume to 200 ml with water

4. Pour back into original beaker and stir

5. Dispense in 20 ml volumes into Universals

6. Label with identity, batch number and expiry date (one year from production)

7. Store at room temperature

8. For use, this 20× Stock solution is diluted 1:19 with reagent grade water to form a 1.5% working solution.

12. Solubilisation and Handling of Compounds

Compounds #1 and #2 are both soluble in water. Each peptide is resuspended in the preferred solvent to a concentration of 1 mg per ml. This stock solution is dispensed in 500 μl volume aliquots in 1 ml bottles, labelled and stored at −85° C. or, less preferably, at −20° C.

13. Production of Standard Positive and ‘Cut-Off’ Sera

Both Standard Positive and Cutoff sera are constituted from the same pool of ovine sera. These sera were obtained from 10 ewes which had (i) been derived from a C. abortus-free flock, as certified under the Premium Sheep Health Scheme; (ii) been experimentally infected with C. abortus during pregnancy; and (iii) had subsequently undergone abortion or the production of heavily infected placentas. The 10 sera were initially tested for reactivity using a variety of tests before being pooled. The production of the kit Standard Positive (a 1 in 1000 dilution in Dako ‘Canadian’ Stabilisation Buffer) and kit Cutoff Standard (a 1 in 7000 dilution in Dako ‘Canadian’ Stabilisation Buffer) are detailed in Appendix 6.

14. Specific Pathogen Free Lamb Sera Used 1N Studies on Specificity

| LAMB | VACCINE ANTIGEN | |
|---|---|---|
| NO. | Strain/Species | Associated condition |

| | | | |
|---|---|---|---|
| 2403 | P787: | C. pecorum | Ovine arthritis |
| 2398 | P787: | C. pecorum | Ovine arthritis |
| 2404 | W73: | C. pecorum | Ovine enteric |
| 2407 | W73: | C. pecorum | Ovine enteric |
| 665 | 84/604: | C. pecorum | Ovine enteric |
| 668 | 84/604: | C. pecorum | Ovine enteric |
| 1 | S45: | Chlamydia suis | Porcine enteric |
| 3 | 84-796: | C.pecorum | Ovine conjunctivitis |
| 4 | 84-796: | C.pecorum | Ovine conjunctivitis |
| 7 | P787: | C pecorum | Ovine arthritis |
| 8 | P787: | C pecorum | Ovine arthritis |

15. Specific Pathogen Free Lamb Sera Used 1N Studies on Sensitivity

| LAMB | VACCINE ANTIGEN | |
|---|---|---|
| NO. | Strain/Species | Associated condition |
| 2401 | S26/3: C. abortus | Ovine abortion |
| 2397 | S26/3: C. abortus | Ovine abortion |
| 2405 | 28/68: C. abortus | Isolated from sheep lungs |
| 2406 | 28/68: C. abortus | Isolated from sheep lungs |
| 662 | A22: C. abortus | Ovine abortion |
| 663 | A22: C. abortus | Ovine abortion |

### EXAMPLES

### Example 1

Effect on Serological Reactivity of Compound Length and Epitopes Additional to the Primary Epitopes

The results of studies on compound length and the addition of ancillary epitopes are shown in Table 1. The data illustrate that the length of compound has a major impact on serological reactivity. They also demonstrate that several epitopes can occur sequentially in the natural sequence of a Variable Segment.

Sequences of compounds used in Table 1

Compound 2-1; Biotin-KGSSIAAD

Compound 2-2; Biotin-KKKKKKGSSIAAD

Compound 2-3; Biotin-KKKKKKKKKKKGSSIAAD

Compound 2-4; Biotin-CLIGVKGSSAAD

Compound 2-5; Biotin-KKKKKGLIGVKGSSIAAD

Compound 2-6; Biotin-AFNLVGLIGVKGSSIAAD

Compound 2/L; Biotin-KASSAAFNLVGLIVKGSSIAADQ

TABLE 1

| Serum | Net Optical Density | | | | | | |
|---|---|---|---|---|---|---|---|
| Dilution | Pep 2-1 | Pep 2-2 | Pep 2-3 | Pep 2-4 | Pep 2-5 | Pep 2-6 | Pep 2/L |
| 1/100 | 0.000 | 1.515 | 2.075 | 2.173 | 2.344 | 2.635 | 2.629 |
| 1/200 | 0.000 | 0.786 | 1.133 | 1.241 | 1/468 | ND | 1.885 |
| 1/400 | 0.000 | 0.385 | 0.540 | 0.572 | 0.771 | 1.185 | 1.107 |
| 1/800 | 0.000 | 0.174 | 0.214 | 0.278 | 0.358 | ND | 0.544 |
| 1/1600 | 0.000 | 0.068 | 0.095 | 0.133 | 0.156 | 0.288 | 0.263 |
| 1/3200 | 0.000 | 0.046 | 0.031 | 0.064 | 0.066 | 0.130 | 0.115 |
| 1/6400 | 0.000 | 0.024 | 0.007 | 0.040 | 0.036 | ND | 0.056 |

Compound 2-1 (an 8-mer) showed no reactivity whatsoever in these studies, while Compound 2-2 (a 13-mer differing from 2-1 only in terms of a 5-mer spacer region between epitope and biotin) showed reactivity which was approximately 70% of that displayed by the 18-mer Compound 2-3 (which differed from 2-2 by containing 5 further lysine residues on the amine side of the epitope). Thus, compound length can be crucial in determining the reactivity of an epitope; these studies indicate that compounds which are more than 13-mer long are preferable. Preferably they are over 18-mer.

### Example 2

Amino Acid Substitution

Table 2 shows the effect of substituting the isoleucine within Epitope 2 with leucine: a small decrease in reactivity resulted, as indicated in the comparisons between Compounds 2-3 with 2-9 and 2-5 with 2-11. Thus, substitutions within the core epitopes described in this invention are possible without too much loss in compound serological reactivity.

Sequences of compounds used in Table 2

Compound 2-3; Biotin-KKKKKKKKKKKGSS I AAD

Compound 2-9; Biotin-KKKKKKKKKKKGSS L AAD

Compound 2-5; Biotin-KKKKKGLIGVKGSS I AAD

Compound 2-11; Biotin-KKKKKGLIGVKGSS L AAD

TABLE 2
Effect of substituting isoleucine with leucine within Epitope 2,
Variable Segment 2 of the MOMP of C. abortus

| | |
|---|---|
| Serum | Net Optical Densities |

| dilution | Pep 2-3 | Pep 2-9 | Pep 2-5 | Pep 2-11 |
|---|---|---|---|---|
| 1/100 | 2.075 | 1.910 | 2.344 | 2.008 |
| 1/200 | 1.133 | 1.075 | 1.468 | 1.230 |
| 1/400 | 0.540 | 0.519 | 0.771 | 0.637 |
| 1/800 | 0.214 | 0.229 | 0.358 | 0.299 |
| 1/1600 | 0.095 | 0.089 | 0.156 | 0.133 |
| 1/3200 | 0.031 | 0.044 | 0.066 | 0.053 |
| 1/6400 | 0.007 | 0.011 | 0.036 | 0.026 |

### Example 3

Specificity of Compounds

Studies on the specificities of the compounds, with or without additional sequences, are summarised in Tables 3 to 5.

### Example 3A

Table 3 shows the specificities of 5 compounds, which do not fall within the scope of the present invention, when tested with sera from sheep which had never experienced infection with C. abortus.

Sequences of the compounds used in Table 3

Compound 1/AX; 20-mer; Biotin-KTITGMGAVPTGTAAANYKT

Compound 1-5; 21-mer; Biotin-KTITGKKVPTGTAAANYKTPT

Compound 1-6; 21-mer; Biotin-KTITGKKKKGTAAANYKTPTD

Compound 2/L; 23-mer; Biotin-KASSAAFNLVGLIVKGSSIAADQ

Compound 2-7; 21-mer; Biotin-KKKKKKKGVKGSSIAADQLPN

The positive reactions of animal #14 with Compounds 1/AX and, more extensively, with Compound 1-5; in the absence of a reaction with Compound 1-6, is presumed to be due to the [V] PTG sequence present on the amine side of the primary epitope. The PTG sequence is found in the VS1 of C. pecorum, enteric form (Kaltenhoeck et al., 1993).

TABLE 3
Specificities of 5 compounds tested with sera from sheep free of
infection with C. abortus

| | Net OD [Gross OD - No compound OD] | | | | |
|---|---|---|---|---|---|
| | Compound | Compound | Compound | Compound | Compound |
| Animal No. | 1/AX | 1-5 | 1-6 | 2/L | 2-7 |
| 5 | — | — | — | 0.285 | — |
| 11 | — | — | — | — | 0.607 |
| 13 | — | — | — | 0.572 | 0.198 |
| 14 | 0.511 | 0.978 | — | — | — |
| 19 | — | 0.747 | 1.316 | — | 0.119 |
| 28 | — | 0.352 | 0.114 | 0.389 | — |
| 29 | — | — | — | — | 0.144 |
| 31 | — | 0.423 | 0.636 | — | — |
| 36 | — | — | 0.500 | 0.128 | — |
| 37 | — | 0.580 | 0.344 | — | — |
| PS;1/1000 | 3.690 | 3.744 | 3.352 | 1.534 | 1.038 |
| PS;1/15000 | 0.493 | 0.452 | 0.354 | 0.098 | 0.059 |

—Signifies lower OD value compared with 1/15000 dilution of Positive Control Serum
PS - Positive Control Serum

The positive reaction of animals #19 and #31 with Compounds 1-5 and, more extensively, with Compound 1-6, in the absence of a reaction with Compound 1/AX, is presumed to be due to the carboxyl end TPT[D] sequence. The TPT sequence is found in (avian) C. psittaci. The additional D is presumed to be responsible for the higher reactivity of Compound 1-6 compared with Compound 1-5. Although TPTD does not occur in C. psittaci, TPTQ does, and Q would constitute an adequate substitute for D, both amino acids having functionalised aliphatic side-chains and relatively high hydrophilicity.

Animals #28 and #37 both displayed a reactivity with Compound 1-5, less so with Compound 1-6 and none with Compound 1/AX. The reasons for this observation are more obscure. The TPT motif, which operated with animals #19 and #31, may have been involved, but why Compound 1-5 showed a greater reactivity than Compound 1-6 is unclear. The region GKKV in 1-5 is given as GKKK in 1-6; these sequences are not found in C. abortus MOMP sequences, but may occur in other infective organisms.

In terms of the two Variable Segment peptides used in this study, Compound 2/L was the only one to react with sera from animals #5, #28 and #36. These reactions are presumably due to all or part of the sequence KASSAAFNLVGLI. From the observations relating to Table 2 and the findings of Kaltenboeck et al. (1997), the KASSA motif is non-epitopic, while LVGLIG and possibly AFN both appear to contribute to the reactivity of compounds. AFN and LVGLIG both occur in (avian) C. psittaci.

Compound 2-7 reacted with sera from animals #11 (strongly), #19 and #29, whereas Compound 2/L did not. These reactions were presumably due to the motif LPN present in Compound 2-7 and also found in C. psittaci.

Both Compound 2/L and Compound 2-7 reacted with serum from animal #13, presumably from dual recognition of the LPN motif in Compound 2-7 and either AFN and/or LVGLIG in Compound 2/L.

### Example 3B

Specificities of Peptides 2-9,2-10, 1B, 1A/1A and 4B/4B as tested with sera from ewes known not to have been infected with C. abortus, and which had shown positive reactivity in previous tests with various forms of VS 1 and VS2.

Compounds

1. Compound 2-9; E A T A L D T S N G V K G S S I A A D Q

2. Compound 2-10; G V K G S S I A A D Q G V K G S S I A A D Q

3. Compound 1B; K T I T G M T G T A A A N Y K T I T G M

4. Compound 1A/1A; T G T A A A N Y K T T G T A A A N Y K T

5. Compound 4B/4B; A T A L D T S N K F A T A L D T S N K F A

Identities of SPF Lamb Antisera

2397/1 Pre-vaccination

2397/1 Vaccinated with S26/3 (C. abortus)

2401/1 Pre-vaccination

2401/2 Vaccinated with S26/3 (C. abortus)

2405/1 Pre-vaccination

2405/2 Vaccinated with 28/28 (C. abortus)

2406/1 Pre-vaccination

2406/2 Vaccinated with 28/68 (C. abortus)

2407/2 Vaccinated with W73 (C. pecorum)

F1/1 Pre-vaccination

F1/2 Vaccinated with S45 (C. suis)

F4/2 Vaccinated with 84-796 (C. pecorum)

F6/2 Vaccinated with Cap. abortion 15 (C. abortus)

F7/2 Vaccinated with P787 (C. pecorum)

F8/2 Vaccinated with P787 (C. pecorum)

TABLE 4

| Animal no. | 2-9 | 2-10 | 1B | 1A/1A | 4B/4B |
|---|---|---|---|---|---|
| A2261 | — | — | — | 0.7 | 0.4 |
| B2123 | — | — | 1.55 | 2.0 | 0.4 |
| 3178 | — | — | 0.7 | — | 0.6 |
| 3201 | — | — | — | — | 1.7 |
| X2895 | — | — | — | — | 0.5 |
| X2899 | — | — | — | — | 0.4 |
| Z148 | — | — | — | — | 0.7 |
| Z2600 | 0.49 | 1.1 | — | — | — |
| Z2611 | — | — | 0.6 | 1.0 | — |
| Z2618 | — | — | — | — | 0.55 |
| Z2646 | — | — | — | 0.4 | 1.0 |
| Z2656 | — | — | — | 0.66 | — |
| Z2708 | 0.4 | 0.55 | — | — | — |
| Z2739 | — | — | — | — | 0.4 |
| Z2680 | — | — | 0.5 | — | — |
| 2948 | 0.4 | 0.5 | — | 0.6 | — |
| 3174 | 1.2 | 1.3 | — | — | — |
| A2332 | 0.9 | 1.0 | — | — | — |
| 2401/2 C.ab | 1.3 | 1.5 | OVER | OVER | |
| 2397/1 | — | — | OVER | OVER | 0.8 |
| 2397/2 C.ab | 3.0 | 3.1 | OVER | OVER | — |
| 2405/2 C.ab | 2.5 | 2.6 | OVER | OVER | — |
| 2406/2 C.ab | 1.2 | 1.6 | OVER | OVER | OVER |
| 2407/2 W73 | — | — | 1.3 | 1.8 | 0.8 |
| F1/1 | — | — | — | — | 0.5 |
| F1/2 S45 | — | — | — | — | 1.2 |
| F4/2 84-796 | — | — | — | 0.4 | — |
| F6/2 Cap.ab | — | — | 0.9 | 0.9 | 0.7 |
| F7/2 P787 | — | — | — | 0.5 | — |
| F8/2 P787 | — | — | — | — | 0.4 |
| 1354R | 0.7 | 0.9 | 2.3 | 2.3 | — |
| 1715T | 0.3 | 0.3 | — | — | — |
| A2213 | — | — | OVER | OVER | OVER |
| Y10-8-14 | — | — | — | 1.2 | — |
| Y10-8-37 | — | — | — | 0.8 | — |

No real differences between 2-9 and 2-10; therefore the VS4 component does not appear to contribute anything. The cross-reactions seen with sera from Z2600, Z2708, 2948, 3174, A2332 are presumably due to the sequence GVKGSSIAADQ.

In contrast, 1B was different from 1A/1A in some respects, with 1A/1A less specific. The cause of this was presumably due to either the TGT or, more likely, the KTTG bridge between the two epitopes.

However, 1B showed reactivity with 2 sera which showed no reactivity with 1A/1A, so this peptide too has cross-reactive elements not found in 1A/1A. The problem area in 1B is presumably related to the KTITGMT sequence.

### Example 4

### Comparison of Compounds for Sensitivity and Specificity

Compounds:

Compound #1: SGTAAANYKTKGTAAANYKT

Compound #2: GKGSSIAADQNKGSSIAADQ

Compound 2-A: GSGSGSGSGSGGVKGSSIAADQ

Compound 2L; KASSAAFNLVGLIVKGSSIAADQ

Compound 2-6: KKKKKKKKKKGSSIAAD

Compound 1/AX: KTITGMGAVPTGTAAANYKT

TABLE 4

| | | | Sensitivity % | | | |
|---|---|---|---|---|---|---|
| | | | Clinical/Micro | | | |
| | Specificity % | | pos'ive | | All infected sheep | |
| Peptide | 1X CO | 2X CO | 1X CO | 2X CO | 1X CO | 2X CO |
| 1/AX | 99.2 | 99.7 | 65.4 | 56.2 | 51.5 | 43.4 |
| #1 | 48.2 | 79.4 | 84.3 | 69.9 | 73.7 | 59.1 |
| 2L | 54.4 | 75.2 | 88.9 | 76.5 | 79.8 | 64.7 |
| 2A | 60.3 | 76.1 | 86.3 | 77.8 | 75.3 | 66.2 |
| 2-6 | 71.0 | 83.1 | 65.4 | 58.2 | 53.5 | 47.0 |
| #2 | 66.8 | 80.9 | 76.5 | 65.3 | 65.7 | 53.0 |
| #1 & #2 | 98.9 | 99.4 | 80.4 | 75.8 | 64.1 | 59.1 |

1X CO = Positivity defined as an OD ≧ the Cutoff OD, the Cutoff standard being a 1 in 10000 dilution of the Standard Positive serum
2X CO = Positivity defined as an OD ≧ 2 times the Cutoff OD, the Cutoff standard being a 1 in 10000 dilution of the Standard Positive serum

Specificity=[Number of true negative animals testing negative/Total number of true negatives]×100

Sensitivity=[Number of true positive animals testing positive/Total number of true positives]×100

553 sera were tested, of which 198 were from animals which had been experimentally infected with Chlamydophila abortus, or vaccinated with this organism. Since only 153 of these animals revealed any evidence of infection subsequently (abortion and/or the excretion of Chlamydophila abortus as detected by culture and/or MZN staining of clinical samples), two forms of determining sensitivity have been used. One accepts all challenged animals to be positive: the other only accepts those animals which showed clinical/microbiological evidence of infection to the positive.

The results obtained illustrate the high degree of specificity and sensitivity obtained when using the compounds according to the present invention. It is also evident that the compounds used, and combinations thereof, clearly provide unique reagents for sensitive assays for the presence of antibodies to C. abortus without displaying false positive reactions with antibodies induced by C. psittaci or C. pecorum.

### REFERENCES

Anderson, I. E., Herring, A. J., Jones, G. E., Low, J. C. and Greig, A. 1995 Veterinary Microbiology 43, 1-12

Andrews, A. H., Goddard, P. C., Wilsmore, A. J. and Dagnall, G. J. R. 1987 Veterinary Record, 120, 238-239

Ball, 1994, Journal of Immunological Methods 171, 37-44

Brade, L., Brunnemann, H., Ernst, M., Fu, Y., Kosma, P., Naher, H., Persson, K., Brade, H. 1994 FEMS Immunology and Medical Microbiology, 8, 2742

Brown, A. S., Amos, M. L., Lavin, M. F., Girjes, A. A., Timms, P. and Woolcock, J. B. 1988 Australian Veterinary Journal, 65, 288-289

Buxton, D. 1986 Veterinary Record. 118, 510-511

Cass, T. and Ligler, F. S. 1998 eds. Immobilised biomolecules in analysis; a practical approach. Oxford University Press Inc., New York

Clarkson, M. J. and Philips, H. L. 1997 Veterinary Journal 153, 307-311

Everett, K. D. E., Bush, R. M. and Andersen, A. A. 1999 International Journal of Systematic Bacteriology 49, 415-440

Fukushi, H. and Hirai, K. 1992 International Journal of Systematic Bacteriology 42, 306-308

Gregorius, K., Mouritsen, S. and Elsner, H. I. Journal of Immunological Methods 181, 65-73

Griffiths, P. C., Plater, J. M., Horigan, M. W., Rose, M. P. M., Venables, C. and Dawson, M. 1996 Journal of Clinical Microbiology, 34, 1512-1518

Harshfield, G. S. 1970 Journal of the American Veterinary Medical Association 156, 466-

Herring, A. J., Tan, T. W., Baxter, S., Inglis, N. F. and Dunbar, S. 1989 FEMS Microbiology Letters 65, 153-158

Holliman, A., Daniel, R. G., Parr, J. G., Griffiths, P. C., Bevan, B. J., Martin, T. C., Hewinson, R. G., Dawson, M. and Munro, R. 1994 Veterinary Record 134, 500-502

Hopkins, J. B., Stephenson, E. H., Storz, J. and Pierson, R. E.1973 Journal of the American Veterinary Medical Association 163, 1157-1161

Jones, G. E., Jones, K. A., Machell, J., Brebner, J., Anderson, I. E. and How, S. 1995 Vaccine, 13, 715-723

Jones, G. E., Low, J.C., Machell, J. and Armstrong, K. 1997 Veterinary Record, 141, 164-168

Kaltenboeck, B., Heard, D., Graves, F. J. and Schmeer, N. 1997 Journal of Clinical Microbiology 35, 2293-2298

Kaltenboeck, B., Kousoulas, K. G. and Storz, J. 1993 Journal of Bacteriology 175, 487-502

Markey, B. K., McNulty, M. S. and Todd, D. 1993 Veterinary Microbiology 36, 233-252

Martin, P. K. 1995 Research in Veterinary Science 58, 193-194

Merrifield, and Barany, 1980 The Peptides: Analysis, Synthesis, Biology Vol.1, Gross and Meinenhofer, eds. Academic Press, New York

Nabeya, M., Kaneko, K., Ogino, H., Nakabayashi, D., Watanabe, T., Murayama, J., Hayashi, K., Fukushi, H., Yamagushi, T., Hirai, K., Inaba, Y. and Matumoto, M. 1991 Veterinary Microbiology 29, 261-265

Modrow S. and Wolf H. 1998 Use of Synthetic Peptides in Microbial Diagnostics in Rapid Detection of Infectious Agents, Specter et al., eds. Plenum Press, N.Y. and London

Narvanen, A., Puolakkainen, M., Hao, W., Kino, K., Imada, M. and Suni, J. 1996 Proceedings of the Third Meeting of the European Society for Chlamydia Research, Vienna. ed. A. Stary, p366

Niveleau, A., Bruno, C., Drouet, E., Brebant, R., Sergeant, A. and Troalen, F. 1995 Journal of Immunological Methods 182, 227-234

Paukku, M., Narvanen, A., Puolakkainen, M., Dreesbach, K., Hao, W., Paavonen, J. and Saikku, P. 1996 Proceedings of the Third Meeting of the European Society for Chlamydia Research, Vienna. ed. A. Stary, p365

Perez-Martinez, J. A. and Storz, J. 1985. Infection and Immunity 50, 905-910

Piercy, D. W. T., Griffiths, P. C. and Teale, C. J.1999 Veterinary Record 144, 126-128

Salti-Montesanto, V., Tsoli, E., Papavassiliou, P, Psarrou, E., Markey, B., Jones, G. E. and Vretou, E. 1997 American Journal of Veterinary Research 58, 228-235

Sanderson, T. P., Andersen, A. A., Miller, L. D., Andrews, J. J., Janke, B. H., Larson, D. L. and Schwartz, K. J. 1994 Journal of Veterinary Diagnostic Investigations 6, 315-320

Shroder, and Lubke, (1966) The Peptides, Vol.1, publ. Academic Press, New York

Spears, P. and Storz, J. 1979. Infection and Immunity 24, 224-232

Stamp, J. T., Watt, J. A. A. and Cockburn, R. B. 1952 Journal of Comparative Pathology 62, 93-101

Stephenson, E. H., Storz, J. and Hopkins, J. B. 1973 American Journal of Veterinary Research, 35, 177-180

Sting, R. and Hafez, H. M. 1992 Zentralblattfur Bakteriologie, 277, 436-445

Wittenbrink, M. M., Schoon, H. A., Schoon, D., Mansfeld, R. and Bisping, W. 1993. Journal of Veterinary Medicine B40, 437-450

Woollen, N., Daniels, E. K., Yeary, T., Leipold, H. W. and Phillips, R. M. 1990 Journal of the American Veterinary Medical Association 197, 600-601

1. A compound having a binding affinity for one or more antibodies specific to Chlamydophila abortus, having the following general formula:
(X)ₘ-[A]-(X)ₙ-[A]ᵥ-(X)ₚ-[A]_{w}-(X)_{q}-[A]_{y}-(X)ᵣ-[A]_{z}-(X)ₛ
wherein
X is a linker molecule;
m, n, p, q, r and s represent the number of linker molecules and are independently any integer between 0 to 15;
v, w, y and z are independently 0 or 1; and
A is independently selected from the following sequences:
i. TAAANYK
ii. GTAAANYK
iii. TAAANYKT
v. GTAAANYKT
v. KGSSIAAD
vi. KGSSIAADQ
wherein the compound does not comprise:
PTGTAAANYK, VKGSSIAADQ, LVGLIG, AFN, LP, TPT, TG, VK;
and the compound is not GSSIAADQ.

2. A compound as claimed in claim 1 consisting of 18 to 24 amino acids.

3. A compound as claimed in claim 1 or 2 consisting of 20 to 23 amino acids.

4. A compound as claimed in claim 1, 2 or 3 wherein X is an amino acid which is considered not to contribute directly to the epitope reactivity.

5. A compound as claimed in claim 4 wherein X is serine, lysine, glycine, asparagine, tyrosine or arginine.

6. A compound as claimed in any one of claims 1 to 5, wherein v is 1, and w, y and z are all 0.

7. A compound as claimed in any one of claims 1 to 6, wherein A is peptide (v) as defined in claim 1 (GTAAANYKT) or is peptide (vi) as defined in claim 1 (KGSSIAADQ).

8. A compound as claimed in any one of claims 1 to 7, which is:
[x]ₙ-GTAAANYKT-[X]ₙ-GTAAANYKT;
wherein X is S (serine) or K (lysine); or
[X]ₙ-KGSSIAADQ-[X]ₙ-KGSSIAADQ;
wherein X is G (glycine) or N (asparagine).

9. A compound as claimed in claim 8 which is:
Compound #1:SGTAAANYKTKGTAAANYKT; or
Compound #2: GKGSSIAADQNKGSSIAADQ

10. A compound as claimed in any one of claims 1 to 9, which is conjugated at the amine terminus with biotin.

11. A compound which is a homologue of a compound as defined in any one claims 1 to 10.

12. A composition comprising one or more compounds as defined in any one of claims 1 to 11.

13. The use of a compound as defined in any of claims 1 to 11 in medicine.

14. The use of a compound as defined in any of claims 1 to 11 in the diagnosis of infection of a subject by C. abortus.

15. The use as claimed in claim 14, wherein the diagnosis is carried out on a biological sample derived from the subject.

16. The use as claimed in claim 14 or 15, wherein the subject is a mammal such as a human, sheep, cattle, goat, pig or horse.

17. The use as claimed in claim 15 or 16, wherein the biological sample is blood, saliva, mucus or other body fluid, tissue or any solubilisable sample which might contain antibodies to C. abortus.

18. The use as claimed in any one of claims 13 to 17, wherein more than one type of compound is used together.

19. The use as claimed in any one of claims 14 to 18, wherein the compounds used are:
[X]ₙ-GTAAANYKT-[X]ₙ-GTAAANYKT;
wherein X is S (serine) or K (lysine) and
[X]ₙ-KGSSIAADQ-[X]ₙ-KGSSIAADQ;
wherein X is G (glycine) or N (asparagine);
wherein n represents independently any integer between 0 to 15.

20. The use as claimed in claim 19, wherein the compounds are:
Compound #1: SGTAAANYKTKGTAAANYKT; and
Compound #2: GKGSSIAADQNKGSSIAADQ.

21. A method of detecting an antibody to C.abortus in a sample of body fluid, comprising the steps of:
contacting the sample with one or more compounds as defined in any one of claims 1 to 11; and
detecting the binding, if any, of said antibody with said compound.

22. A method as claimed in claim 21, wherein more than one type of compound is used.

23. A method as claimed in claim 21 or 22, wherein the sample is contacted with the following compounds:
[X]ₙ-GTAAANYKT-[X]ₙ-GTAAANYKT;
wherein X is S (serine) or K (lysine)
and
[X]ₙ-KGSSIAADQ-[X]ₙ-KGSSIAADQ;
wherein X is G (glycine) or N (asparagine);
wherein n represents independently any integer between 0 to 15.

24. A method as claimed in claim 23, wherein the compounds are:
Compound #1:SGTAAANYKTKGTAAANYKT; and
Compound #2:GKGSSIAADQNKGSSIAADQ.

25. A method as claimed in any one of claims 21 to 24, wherein enzyme-linked immunosorbent assay (ELISA) (indirect and competitive forms), haemagglutination assay, radioimmunoassay, latex agglutination assay, fluorescent polarising assay, biosensors of various types, including amperometric, electrochemical and other means for detecting antibodies or complexes thereof, including immunological complexes, and others, are used.

26. A diagnostic kit for infection by C.abortus, comprising:
a compound as defined in any one of claims 1 to 11; and
at least one reagent for detecting the binding of an antibody to C. abortus and the compound.

27. A compound or a composition as defined in any of claims 1 to 12 for use in medicine.
